(19) United States
(12) Patent Application Publication
(10) Pub. No.: US 2026/0151506 A1
(43) Pub. Date: Jun. 4, 2026

(54) MEDICINAL PREPARATION FOR COMBINED GENE-DIRECTED ENZYME PRODRUG THERAPY AND IMMUNOTHERAPY OF SOLID MALIGNANT TUMOURS (EMBODIMENTS)

(71) Applicant: OBSHCHESTVO S OGRANICHENNOJ OTVETSTVENNOSTYU "GENNAYA KHIRURGIYA", Moscow (RU)

(72) Inventors: Irina Vasilevna ALEKSEENKO, Moscow (RU); Evgenij Davidovich SVERDLOV, Moscow (RU); Maksim Olegovich KOKSHAROV, Yekaterinburg (RU); Evgenij Valerevich SNEZHKOV, Yekaterinburg (RU)

(21) Appl. No.: 19/106,521

(22) PCT Filed: Apr. 14, 2023
(86) PCT No.: PCT/RU2023/050090

§ 371 Date: Feb. 25, 2025

(30) Foreign Application Priority Data
Aug. 21, 2022 (RU) 2022123323

(51) Int. Cl. A61K 48/00; A61K 38/19; A61K 38/45; A61K 47/02; A61K 47/22; A61K 47/26; A61K 47/60; A61K 47/64; A61P 35/00

(52) U.S. Cl. CPC A61K 48/005; A61K 38/193; A61K 38/45; A61K 47/02; A61K 47/22; A61K 47/26; A61K 47/60; A61K 47/645; A61K 48/0075; A61P 35/00; C12Y 207/01021

(57) ABSTRACT

The present invention relates to the field of biotechnology, medicine, in particular, oncology and pharmaceuticals, and is directed to ready-to-use dosage forms of a medicinal product for combination gene-directed enzyme prodrug therapy—immune therapy of human solid malignant neoplasms.

Two variants of two-component dosage forms of the medicinal product are proposed. In the first variant, the composition of the medicinal product includes the first and second containers, the first container containing the first component containing the pCMV-HSVtk-hGM-CSF therapeutic plasmid DNA in a therapeutically effective amount in the form of a frozen aqueous solution, and the second container containing the second component containing the polyethyleneimine-polyethylene glycol-TAT peptide block copolymer in a therapeutically effective amount in the form of a frozen aqueous solution. A ready-to-use solution for injection, intended for administration to a patient, is prepared ex tempore by thawing the referenced two components and mixing them.

In the second variant, the composition of the medicinal product includes the first and second containers, the first container containing the first component containing the pCMV-HSVtk-hGM-CSF therapeutic plasmid DNA in a therapeutically effective amount in the form of a lyophilisate, and the second container containing the second component containing the polyethyleneimine-polyethylene glycol-TAT peptide block copolymer in a therapeutically effective amount in the form of a frozen aqueous solution. A ready-to-use solution for injection intended for administration to a patient is prepared ex tempore by reconstituting the first component by adding water, thawing the second component and mixing them.

The proposed dosage forms can be used for the treatment of human solid malignant neoplasms. The two proposed dosage forms demonstrate high stability and retain their therapeutic efficacy for at least 18 months.

### CROSS-REFERENCE TO RELATED APPLICATIONS

This application is the U.S. national phase of International Application No. PCT/RU2023/050090 filed Apr. 14, 2023, which designated the U.S. and claims priority to RU 2022123323 filed Aug. 31, 2022, the entire contents of each of which are hereby incorporated by reference.

### TECHNICAL FIELD

The present invention relates to the field of biotechnology, medicine, in particular, oncology and pharmaceuticals, and is directed to dosage forms of a medicinal product for combination gene-directed enzyme prodrug therapy—immune therapy of solid malignant neoplasms.

The disclosed dosage forms can be used to treat various solid malignant neoplasms in humans. The proposed dosage forms demonstrate high stability and retain their therapeutic efficacy for at least 18 months.

### BACKGROUND ART

Cancer (malignant neoplasms) is one of the most serious problems in medicine: it ranks second in terms of mortality in the world, and cancer incidence is on the rise.

Oncological diseases combine both the complexity of the cellular organization and the properties of a complex growing system capable of adapting to external influences, including medicaments, due to microheterogeneity within such tumors. The heterogeneity of cancerous tumors lies in the fact that each cancer cell is different from all other cells of the same tumor, and the tumor in one patient is different from that of the same type in another one. Therefore, among many cells in a tumor, there are always cells resistant to therapeutic effects and capable of giving rise to a new, resistant to this effect, tumor clone. As a result, it becomes apparent that it is rather difficult to unambiguously identify any universal key genes/gene products which can be used as targets for anticancer therapy.

Classical chemotherapy as a standard treatment for cancer patients, created over several decades, has proven effective in slowing down the development of various types of malignant neoplasms. However, the therapeutic index of classical chemotherapy is often low due to the lack of specificity of medicaments to cancer cells compared to normal ones and due to the difficulty in achieving therapeutic intratumoral concentrations in the absence of systemic toxicity. These problems are particularly relevant for solid tumors, as poor neovascularization and associated necrotic areas often render such tumors relatively resistant to efficient uptake of systemically delivered medicaments. In connection with the current situation around the world, the search and implementation of novel strategies for cancer therapy is underway.

One such strategy is gene-directed enzyme prodrug therapy (GDEPT) (Sverdlov E. D. Curr. Gene Ther. 2011, 11(6), p. 501-531). This approach is also known as “suicidal gene therapy” or “gene surgery.” The approach of gene surgery is to deliver genes encoding an enzyme which is able to convert its systemically administered substrate into a highly toxic agent in the cells where it is expressed, to target cancer cells. This strategy is similar to chemotherapy, however, unlike it, the toxin is formed inside the cancer cell in the case of gene surgery, so the toxic effect on normal cells of the body is minimized.

The GDEPT approach makes it possible to kill cancer cells regardless of their genesis and properties. Another important advantage of gene surgery is that even a small number of cells expressing the transgene is sufficient to cause the death of a large number of tumor cells. The toxin formed in cells expressing the transgene can be released therefrom and penetrate into neighboring cells which have not received the transgene, causing their death. This phenomenon is called the bystander effect.

Currently, the most studied system for conducting GDEPT is the herpes simplex virus thymidine kinase (HSVtk)/ganciclovir (GCV) combination, which has reached the late stages of clinical trials, during which its efficacy and safety have been shown (Immonen A. et al. Mol. Ther. 2004, 10(5), p. 967-972; Westphal M. et al. Lancet Oncol. 2013, 14(9), p. 823-833). Ganciclovir is a registered medicinal product and is commercially available, for example, as Cymeven®. Delivery of the therapeutic gene encoding a thymidine kinase enzyme, for example, herpes simplex virus thymidine kinase (HSVtk), as part of an expression construct to tumor cells ensures the production of the HSVtk enzyme inside these cells. Ganciclovir is a guanosine analog and a prodrug administered systemically. Ganciclovir is phosphorylated to ganciclovir monophosphate under the action of the thymidine kinase enzyme in the tumor cell expressing HSVtk. Then, under the action of intracellular kinases, ganciclovir monophosphate is sequentially converted into ganciclovir diphosphate and ganciclovir triphosphate. Acting as a substrate and incorporating into DNA, ganciclovir triphosphate competitively inhibits DNA polymerase, which leads to suppression of DNA synthesis due to inhibition of DNA chain elongation and to single DNA breaks and, ultimately, to the death of tumor cells. In addition, the newly formed ganciclovir triphosphate can leave the tumor cells which it was formed in and penetrate into neighboring tumor cells, leading to their death (the bystander effect mentioned above). Cell death mediated by the HSVtk/ganciclovir combination can be caused by various mechanisms, namely: apoptosis and necrosis, as well as immunogenic cell death (ICD) with the release of tumor-specific antigens and damage-associated molecular patterns (DAMP), which leads to infiltration of the tumor with various antigen presenting cells, including dendritic and T cells (Sverdlov E. D. Curr. Gene Ther. 2011, 11(6), p. 501-531).

Due to the fact that some tumor cells (or the tumor as a whole) can be resistant to some toxic agents used in GDEPT systems, researchers develop approaches to improve the efficacy of gene surgery. One such approach is the combination of gene surgery with immunotherapy. This approach is sometimes referred to as gene-immune therapy.

Tumor cells in the early stages of carcinogenesis are known to be almost indistinguishable in their molecular composition from the cells of the surrounding tissue; they cannot be recognized and destroyed by the immune system, being non-immunogenic. As the tumor grows due to the accumulation of mutations in DNA, so-called neoantigens are formed, which can be presented by dendritic cells to T-helper cells, which are activators of specific immunity to tumors. The appearance of neoantigens occurs with a delay, which allows tumor cells to implement mechanisms of protection against the immune system, in particular, to create a microenvironment that inhibits specific antitumor immunity.

Many members of the cytokine family are able to mobilize the immune system of patients, contributing to the development of a specific antitumor response. For example, in Imlygic gene-therapy anticancer drugs (Amgen Inc., USA), an approach based on increased production of the granulocyte-macrophage colony-stimulating factor (GM-CSF) cytokine was used to enhance the presentation of tumor antigens by dendritic cells. Studies have shown that cell-based vaccines containing GM-CSF provide a stronger antitumor response in animals than vaccines including other cytokines (Dranoff G, Jaffee E et al. (1993) Proc. Natl. Acad. Sci. USA 90:3539-43).

Granulocyte-macrophage colony-stimulating factor (GM-CSF) belongs to the family of hematopoietic cytokines. The biological activity of GM-CSF is mediated by its binding to a heteromeric receptor which is expressed on monocytes, macrophages, granulocytes, lymphocytes, endothelial cells, and alveolar epithelial cells. After specific binding of GM-CSF to the receptor, signal transduction along the JAK2 pathway occurs, resulting in a functional response. GM-CSF stimulates the proliferation and differentiation of granulocytes and monocytes, enhances the phagocytic and cytotoxic activity of granulocytes and eosinophils, stimulates the proliferation, maturation and differentiation of dendritic cells, and plays an important role in the recruitment and activation of antigen-presenting cells.

The expression of GM-CSF in tumor cells is known to increase the tumor's immunogenicity, thereby contributing to its elimination and ensuring the development of an antitumor response. In vivo studies have shown that the efficacy of the HSVtk/GCV system increases dramatically if the HSVtk gene is introduced into tumor cells together with the GM-CSF gene (Jones R. K., Pope I. M., et al. (2000) Cancer Gene Ther. 7:1519-28; Finocchiaro L. M., Fiszman G. L., et al. (2008) Cancer Gene Ther. 15:165-72).

The superiority of the combination therapy combining the administration of GM-CSF and the HSVtk/GCV system over gene surgery involving only the administration of the HSVtk/GCV system has been demonstrated in ex vivo and in vivo studies.

The antitumor effect of GM-CSF in combination with the HSVtk/GCV system is believed to be due to the following mechanism: when the HSVtk and GM-CSF genes enter cancer cells simultaneously, cancer cells will die due to the functioning of the HSVtk/GCV system and tumor antigens will be released therefrom. Tumor antigens will be effectively presented by GM-CSF-activated antigen-presenting cells to T-cells of the immune system, providing activation of specific antitumor immunity, resulting in increased tumor cell death and reduced probability of metastases. Attracting antigen-presenting cells to the tumor and their activation occurs through the synthesized GM-CSF. As a result, the effective presentation of tumor antigens by GM-CSF-activated antigen-presenting cells to T-cells of the immune system and, as a result, activation of specific antitumor immunity occur.

One of the important elements of the success of the gene therapy of cancer is an adequate system for the delivery and expression of therapeutic genes, since these genes must work in cancer cells and must not work in normal cells of the body. Viral and non-viral systems are used to deliver therapeutic constructs to the patient's tumor cells. The main disadvantage of non-viral delivery systems compared to viral ones is their lower transfection efficacy. At the same time, non-viral delivery systems have a number of merits, such as high packing capacity, low immunogenicity, high safety, and the plausibility of cost-effective production (Yue Y. and Wu C. (2013) Biomaterials Science 1:152-170).

Currently, polycations are widely used as non-viral DNA delivery systems for gene therapy. DNA complexes with polycations, or polyplexes, are able to compact DNA and protect it from external factors. Due to certain peculiarities in their chemical structure, polyplexes provide for the possibility of modification, in particular, by covalent attachment of molecules providing directed (targeted) interaction with the target. Polyplexes are able to overcome endosomal membranes and enter the cytosol; they are non-pathogenic, almost non-immunogenic, and non-toxic. The relatively low cost of production makes them very attractive to pharmaceutical companies, and the reproducibility of the production of polyplexes with covalently attached ligands is quite high (Kang H. C., Lee M., et al. (2005) Crit. Rev. Eukaryot. Gene Expr. 15:317-42). Polyplexes can be used for both systemic and local administration: intratumorally into the lungs, various tumors, the brain, mammary glands and oviducts. Polyplexes have been shown to be effective in gene therapy for hepatocellular carcinoma (Iwai M, Harada Y et al. (2002) Biochem. Biophys. Res. Commun. 291:48-54) and disseminated pancreatic cancer (Aoki K, Furuhata S et al. (2001) Gene Ther. 8:508-14).

This approach of combination GDEPT-immune therapy using a polycationic delivery system was successfully implemented, for example, in the pharmaceutical composition described in RU 2 575 077. The pharmaceutical composition comprises the following active substances: 1) a nucleic acid encoding two therapeutic genes (killer gene and immunomodulatory gene) and regulatory elements necessary for their expression; 2) a polymeric polycationic carrier consisting of three components: polyethylene glycol (PEG), polyethyleneimine (PEI) and TAT peptide. The nucleic acid is a high purity supercoiled form of the plasmid with the general formula pCMV-HSVtk-hGM-CSF and encodes herpes simplex virus thymidine kinase (HSVtk) and human granulocyte-macrophage colony-stimulating factor (GM-CSF) under the control of the cytomegalovirus (CMV) promoter (DNA control region). The polycationic carrier is a block copolymer (BC) of PEI and PEG with a conjugated TAT peptide (together PPT). The TAT peptide, characterized by the sequence GRKKKRRQRC, is a fragment of the TAT protein and enhances the ability of the block copolymer, which it is included in, to penetrate into cells and ensure the delivery of the plasmid into tumor cells (Ulasov A V, Khramtsov Y V et al. (2011) Mol. Ther. 19:103-12; Rudolph C, Plank C et al. (2003) J. Biol. Chem. 278:11411-8).

The recombinant plasmid DNA, described in RU 2 575 077, consists of the following components: a fragment of the promoter site (581 nucleotide pairs), which drives the transcription of the genes; the HSVtk gene, upon expression of which the HSVtk form is synthesized, consisting of 374 amino acid residues; an internal ribosome entry site (IRES) allowing expression of both therapeutic genes from a single vector (Mizuguchi, Xu et al. (2000) Mol. Ther. 1:376-82); the hGM-CSF or mGM-CSF gene, whose expression produces the hGM-CSF (human) or mGM-CSF (mouse) form, respectively; the SV40 polyadenylation signal and transcription terminator that provide the synthesis of mature RNA from the genes. Since the granulocyte-macrophage colony-stimulating factor protein is species-specific (Shi Y, Liu C H et al. (2006) Cell Res. 16:126-33), and animals with grafted tumors (xenografts) are most often used to test the efficacy of gene therapy constructs, RU 2 575 077 discloses two variants of the construct, one containing the mouse GM-CSF gene for mouse model experiments, and the other containing the human GM-CSF gene for clinical trials and direct use in patients. In addition, the recombinant plasmid DNA described in RU 2 575 077 additionally contains auxiliary regions necessary for the efficient biosynthesis of plasmid DNA on an industrial scale in the cells of the E. coli producer strain. These auxiliary regions include, in particular, f1 ori, Co1E1 ori, the ampicillin resistance selective marker AmpR.

Upon penetration of the molecules of the pCMV-HSVtk-hGM-CSF plasmid into tumor cells, the HSVtk enzyme is synthesized, which is capable of phosphorylating the nucleoside analogue of ganciclovir administered systemically to ganciclovir monophosphate, which is then phosphorylated by endogenous cellular kinases to a toxic form, ganciclovir triphosphate. During cell division, ganciclovir triphosphate is incorporated into the newly synthesized DNA chain and interrupts its further synthesis, causing cell death. Also, when the plasmid molecules penetrate into tumor cells, the GM-CSF protein is synthesized, which is secreted into the extracellular space and stimulates the growth, development and differentiation of granulocytes and antigen-presenting cells, attracting them to the tumor growth zone to provide the induction of a specific antitumor immune response. The mechanism of induction of antitumor immunity by GM-CSF includes the direct involvement of natural killer cells and antigen-presenting cells, for example, dendritic cells in the tumor growth zone, which leads to the activation of T cells. Thus, due to the activity of herpes simplex virus thymidine kinase, the administered ganciclovir causes the death of tumor cells that release tumor antigens, which are presented to T cells of the immune system by antigen-presenting cells, activated by GM-CSF. Activation of the adaptive immune response caused by the medicinal product can provide local and systemic immune-mediated destruction of distant tumors, thereby providing an additional effect of antitumor vaccination, and lead to the development of immunological memory, as well as long-term tumor growth control using immune surveillance.

The pharmaceutical composition disclosed in RU 2 575 077 contains the following excipients: 5 mM HEPES (4-(2-hydroxyethyl)-1-piperazine ethanesulfonic acid), pH 7.4, 12.5 mM borate buffer, pH 7.5, and 5% glucose.

In model experiments in mice, the high efficacy of the GDEPT-immune therapy combination system based on the use of the pCMV-HSVtk-GM-CSF plasmid in combination with systemic administration of ganciclovir against a wide range of solid malignant neoplasms was shown in RU 2 575 077. In particular, the efficacy of this system was demonstrated against sarcoma, colon adenocarcinoma, cervical cancer, laryngeal carcinoma, and fibrosarcoma.

One of the main problems in the use of gene therapy medicinal products in medicine is their storage stability. As mentioned above, gene therapy medicinal products used in clinical practice can be divided into viral and non-viral ones according to the method of their delivery.

Although non-viral gene therapy medicinal products have a number of advantages over viral ones, such as low toxicity, low pathogenicity and low immunogenicity, lower production costs, greater ease of manufacture and/or modification in a certain way to achieve high specificity, both medicinal products are not stable at storage. The stability of such medicinal products is achieved only at negative temperatures and often requires storage at −70° C., while storage temperature abuse may lead to a complete loss of drug activity.

Finished dosage forms of gene therapy medicinal products must meet certain requirements, in particular:
1) the production process should be technological;
2) any finished dosage form must be stable during storage and transportation;
3) it is desirable that the shelf life is long; and
4) any finished dosage form should be characterized by ease of preparation for use and ease of use itself.

One of the approaches to the development of dosage forms for gene therapy is obtaining lyophilized medicaments. E.g., WO 2009/021017 describes the successful development of a lyophilized composition containing an excipient (filler), a nucleic acid, and a cationic lipopolymer. The referenced lipopolymer contains a cationic polymer-based backbone with cholesterol molecules and polyethylene glycol molecules covalently attached to it. However, this approach is generally not suitable for the lyophilization of DNA complexes with synthetic vectors, since this approach changes the physicochemical properties, which leads to aggregation and loss of transfection ability when the lyophilized composition is reconstituted with a solvent. The use of cryoprotectants, such as various sugars, dextrans or polyethylene glycol, may lead to improved stability; however, the effect achieved varies and depends not only on the specific cryoprotectant used, but also on the type of a synthetic vector used.

Another important factor affecting the possibility of using lyophilization to obtain dosage forms for gene therapy is that when using even those sugars which are the most effective lyoprotectants, a very high sugar/DNA molar ratio (usually above 1000:1) is required for ensuring stability. As a result, the lyophilized preparation often requires a very high degree of dilution upon reconstitution to achieve acceptable osmolarity (ideally, isotonicity), resulting in a dramatic decrease in the DNA concentration in the reconstituted preparation compared to the original composition subjected to lyophilization. For many preparations with cationic vectors as carriers, the final DNA concentration can be approximately 0.1-0.2 mg/ml and below, which creates certain difficulties in clinical use, since such volumes may be required to administer optimal doses, which are incompatible with the local route of administration. WO 2009/021017 reports this pharmaceutical limitation to be one of the main factors behind the suboptimal performance of synthetic gene delivery systems in human clinical trials.

Thus, in this field of art there is a need to develop new dosage forms of gene therapy medicinal products, in particular, a medicinal product for combination GDEPT-immune therapy based on the therapeutic pCMV-HSVtk-GM-CSF plasmid, combining the possibility of long-term storage under relatively non-stringent conditions, the manufacturability of the technological process, the convenience of preparing the medicinal product for administration to a patient in need of treatment, and the convenience of administration itself.

### DISCLOSURE OF INVENTION

The present invention relates to a two-component medicinal product for combination gene-directed enzyme prodrug therapy—immune therapy of solid malignant neoplasms, wherein a ready-to-use solution for injection intended for administration to a patient is obtained by mixing the first component with the second component. The first component is an aqueous solution stored frozen until use, which contains, as an active substance, pCMV-HSVtk-hGM-CSF supercoiled plasmid DNA, obtained by biosynthesis in E. coli cells and having a direct therapeutic effect. The first component contains the active substance in a therapeutically effective amount. The second component is an aqueous solution stored frozen until use, which contains as an active substance a covalently bound block copolymer of polyethyleneimine-polyethylene glycol-TAT peptide (BC PPT) chemically synthesized in an aqueous phase, which ensures the penetration of plasmid DNA into the cell. The second component contains the active substance in a therapeutically effective amount.

The present invention also relates to a two-component medicinal product for combination gene-directed enzyme prodrug therapy—immune therapy of solid malignant neoplasms, and a ready-to-use solution for injection intended for administration to a patient is obtained by reconstituting the first component by adding water and then mixing with the second component. The first component is a lyophilizate, stored at a temperature not exceeding 4° C., obtained from an aqueous solution containing pCMV-HSVtk-hGM-CSF supercoiled plasmid DNA as an active substance, obtained by biosynthesis in E. coli cells and having a direct therapeutic effect. The first component contains the active substance in a therapeutically effective amount. The second component is an aqueous solution stored frozen until use, which contains, as an active substance, a covalently bound BC PPT chemically synthesized in an aqueous phase, which ensures the penetration of plasmid DNA into the cell. The second component contains the active substance in a therapeutically effective amount.

In both variants of the medicinal product, the ready-to-use solution for injection intended for administration to the patient is prepared ex tempore, i.e. immediately prior to administration to a patient in need of treatment for a solid cancer.

The mechanism of action of the claimed medicinal products is based on the delivery of therapeutic genes HSVtk and hGM-CSF into tumor cells using a non-viral vector, which is a PEG-PEI-TAT (PPT) block copolymer, by injection, preferably by intratumoral administration in order to ensure the production of the HSVtk enzyme and the hGM-CSF cytokine.

The technical result provided by the invention is, in particular, in that the proposed dosage forms demonstrate high stability and retain their therapeutic efficacy for at least 18 months.

Thus, in the first aspect, the claimed invention relates to a medicinal product for the treatment of solid malignant neoplasms by the method of combination gene-directed enzyme prodrug therapy—immune therapy, containing two hermetically sealed containers, wherein:
the first container contains the first component containing pCMV-HSVtk-hGM-CSF supercoiled plasmid DNA in a therapeutically effective amount, at least one pharmaceutically acceptable additive and water, and
the second container contains the second component containing PEG-PEI-TAT peptide block copolymer in a therapeutically effective amount, at least one pharmaceutically acceptable additive and water,
wherein the contents of each of the containers is sterile, and the containers are stored at a temperature not higher than −18° C.

In one embodiment, the medicinal product is characterized in that:
the first component contains:
   pCMV-HSVtk-hGM-CSF supercoiled plasmid DNA—approximately 150-170 μg,
   dextrose—approximately 100 mg,
   HEPES—approximately 2.38 mg, and
   water—to a final volume of 1.0 ml,
the second component contains:
   PEG-PEI-TAT peptide block copolymer—approximately 0.63-0.74 mg (on the PEI basis),
   sodium tetraborate—approximately 2.38 mg, and
   water—to a final volume of 1.0 ml.

In one of the embodiments of the medicinal product, the ready-to-use solution for injection is obtained by thawing the contents of the referenced first and second containers and mixing them.

In yet another embodiment, the ready-to-use solution for injection is administered intratumorally.

In a further embodiment, the solid cancers are selected from the group comprising: lung cancers, pancreatic cancers, melanoma, fibrosarcoma, sarcoma, head and neck cancers.

In one embodiment of the medicinal product, the PEG-PEI-TAT peptide block copolymer has a PEG/PEI molar ratio of about 1.0 to 3.0 and a TAT/PEI molar ratio of about 0.8 to 1.5. In a preferred embodiment, the PEG-PEI-TAT peptide block copolymer has a PEG/PEI molar ratio of about 2.0 to 2.5.

In yet another embodiment, the medicinal product is characterized in that:
the first component contains:
   pCMV-HSVtk-hGM-CSF supercoiled plasmid DNA—approximately 160 μg,
   dextrose—approximately 100 mg,
   HEPES—approximately 2.38 mg, and
   water—to a final volume of 1.0 ml,
the second component contains:
   PEG-PEI-TAT peptide block copolymer—approximately 0.69 mg (on the PEI basis);
   sodium tetraborate—approximately 2.38 mg, and
   water—to a final volume of 1.0 ml,
wherein the PEG-PEI-TAT peptide block copolymer has a PEG/PEI molar ratio of approximately 2.0.

In the second aspect, the present invention relates to a medicinal product for the treatment of solid malignant neoplasms by combination gene-directed enzyme prodrug therapy—immune therapy, containing two hermetically sealed containers, wherein:
the first container contains the first component, which is a lyophilizate containing pCMV-HSVtk-hGM-CSF supercoiled plasmid DNA in a therapeutically effective amount and at least one pharmaceutically acceptable additive, and
the second container contains the second component containing the PEG-PEI-TAT peptide block copolymer in a therapeutically effective amount, at least one pharmaceutically acceptable additive and water,
wherein the contents of each of the containers is sterile, and the first container is stored at a temperature not higher than 4° C., and the second container is stored at a temperature not higher than −18° C.

In one of the embodiments, the medicinal product is characterized in that:
the first component, which is a lyophilizate, has been obtained by lyophilization of a solution containing:
   pCMV-HSVtk-hGM-CSF supercoiled plasmid DNA—approximately 150-170 μg,
   dextrose—approximately 100 mg,
   HEPES—approximately 2.38 mg, and
   water—to a final volume of 1.0 ml,
the second component contains:
   PEG-PEI-TAT peptide block copolymer—approximately 0.63-0.74 mg (on the PEI basis);
   sodium tetraborate—approximately 2.38 mg, and
   water—to a final volume of 1.0 ml.

In one of the embodiments of the medicinal product, the ready-to-use solution for injection is obtained by reconstituting the contents of the first container by adding 1.0 ml of water, thawing the contents of the second container and mixing them.

In the next embodiment, the ready-to-use solution for injection is administered intratumorally.

In yet another embodiment, the solid cancers are selected from the group comprising: lung cancers, pancreatic cancers, melanoma, fibrosarcoma, sarcoma, head and neck cancers.

In another embodiment of the medicinal product, the PEG-PEI-TAT peptide block copolymer is characterized by a PEG/PEI molar ratio of about 1.0 to 3.0 and a TAT/PEI molar ratio of about 0.8 to 1.5. In a preferred embodiment, the PEG-PEI-TAT peptide block copolymer has a PEG/PEI molar ratio of about 2.0 to 2.5.

In yet another embodiment, the medicinal product is characterized in that:
the first component, which is a lyophilisate, has been obtained by lyophilization of a solution containing:
   pCMV-HSVtk-hGM-CSF supercoiled plasmid DNA—approximately 160 μg,
   dextrose—approximately 100 mg,
   HEPES—approximately 2.38 mg, and
   water—to a final volume of 1.0 ml,
and the second component contains:
   PEG-PEI-TAT peptide block copolymer—approximately 0.69 mg (on the PEI basis);
   sodium tetraborate—approximately 2.38 mg, and
   water—to a final volume of 1.0 ml,
wherein the PEG-PEI-TAT peptide block copolymer has a PEG/PEI molar ratio of approximately 2.0.

### BRIEF DESCRIPTION OF DRAWINGS

FIG. 1—Structure of the expression cassette of the pCMV-HSVtk-hGM-CSF supercoiled plasmid DNA. CMV—cytomegalovirus promoter; HSVtk—herpes simplex virus thymidine kinase gene; hGM-CSF—human granulocyte-macrophage colony-stimulating factor gene; IRES—encephalomyocarditis virus internal ribosome entry site; SV40 polyA—SV40 polyadenylation signal.

### DESCRIPTION OF EMBODIMENTS

In numerous preliminary experiments, the inventors of the present invention investigated various dosage forms of the medicinal product for the combination gene-directed enzyme prodrug therapy—immune therapy of solid malignancies. These studies included both liquid (in the form of one-component or two-component systems) and lyophilized (both fully and partially) dosage forms. Based on the results of their studies, the inventors concluded that it was necessary to use two-component systems, since one-component systems (both liquid and lyophilized) did not provide the necessary stability of the medicinal product. In addition, the use of lyophilization in relation to one of the active substances of the medicinal product, namely, PPT, inevitably led to unsatisfactory results. Consequently, for the purposes of further development of dosage forms of the medicinal product for the combination gene-directed enzyme prodrug therapy—immune therapy of solid malignant neoplasms, the inventors have opted for two-component systems, where the first component containing the therapeutic plasmid as an active substance can be either a frozen solution or a lyophilizate, while the second component containing PPT as an active substance must necessarily be a frozen solution.

Thus, in the first aspect of the invention, the medicinal product for the combination GDEPT-immune therapy of solid malignant neoplasms contains two hermetically sealed containers, the contents of the first container (first component) being a frozen solution containing high purity pCMV-HSVtk-hGM-CSF supercoiled plasmid DNA (pDNA) as an active substance.

The contents of the second container (the second component) is a frozen solution containing PPT as an active substance. The PPT block copolymer is a covalently bound copolymer chemically synthesized in an aqueous phase, which includes: PEG—modified polyethylene glycol MAL-dPEG®₂₄-NHS ester, PEI—linear polyethyleneimine 25000, and TAT peptide. The ready-to-use solution for injection, preferably administered intratumorally, is obtained ex tempore by mixing the first component with the second component after their preliminary thawing. Combining the two solutions results in the formation of polyplexes (complexes of the pCMV-HSVtk-hGM-CSF plasmid DNA with the PPT block copolymer) capable of penetrating into tumor cells and inducing the synthesis of two therapeutic proteins HSVtk and hGM-CSF therein.

In the second aspect of the invention, the medicinal product for the combination GDEPT-immune therapy of solid malignant neoplasms contains two hermetically sealed containers, the contents of the first container (first component) being a lyophilisate containing high purity pCMV-HSVtk-hGM-CSF supercoiled plasmid DNA (pDNA) as an active substance.

The contents of the second container (second component) is a frozen solution containing PPT as an active substance.

In the second aspect of the invention, the ready-to-use solution for injections, preferably administered intratumorally, is obtained ex tempore by reconstituting the first component by adding water to the original volume and then mixing with the second component, after its preliminary thawing. Combining the two solutions results in the formation of polyplexes (complexes of the pCMV-HSVtk-hGM-CSF plasmid DNA with the PPT block copolymer) capable of penetrating into tumor cells and inducing the synthesis of two therapeutic proteins HSVtk and hGM-CSF therein.

Structure and Properties of pCMV-HSVtk-hGM-CSF Plasmid DNA

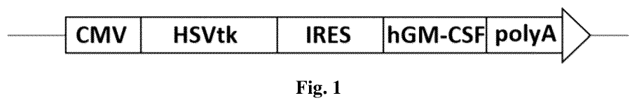

The pCMV-HSVtk-hGM-CSF plasmid contains an expression cassette consisting of two therapeutic genes, namely, the herpes simplex virus thymidine kinase gene HSVtk and the human granulocyte-macrophage colony-stimulating factor gene hGM-CSF, linked through an IRES element under the control of the cytomegalovirus promoter CMV (FIG. 1).

The pCMV-HSVtk-hGM-CSF supercoiled pDNA is produced by biosynthesis in Escherichia coli host cells. Any suitable strain of E. coli can be used as the host cell, as is well known to those skilled in the art. In the present invention, the E. coli DH-5α strain transformed with the pCMV-HSVtk-hGM-CSF plasmid, specifically E. coli DH-5a/pCMV-HSVtk-hGM-CSF, is preferably used.

When the pCMV-HSVtk-hGM-CSF plasmid molecules penetrate into tumor cells, the HSVtk enzyme is synthesized, which is capable of phosphorylating the systemically administered nucleoside analogue—ganciclovir to ganciclovir monophosphate, which is then phosphorylated by cell kinases to its toxic form, ganciclovir triphosphate. Also, when the plasmid molecules penetrate into tumor cells, the human GM-CSF protein is synthesized, which is secreted into the extracellular space and stimulates the growth, development and differentiation of granulocytes and antigen-presenting cells, attracting them to the tumor growth zone to provide the induction of a specific antitumor immune response.

The medicinal product of the present invention contains the pCMV-HSVtk-hGM-CSF supercoiled plasmid DNA in an amount of approximately 150-170 μg.

### Structure and Properties of PPT Block Copolymer (BC PPT)

To obtain BC PPT, the present invention uses modified polyethylene glycol MAL-dPEG®24-NHS ester (PEG):

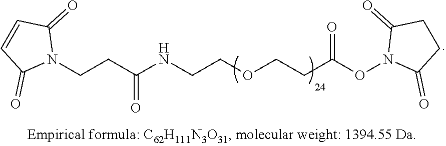

To obtain BC PPT, the present invention uses linear polyethyleneimine 25000 (PEI):

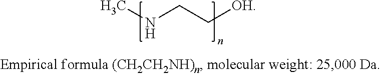

To obtain BC PPT, the present invention uses the TAT peptide having the following amino acid sequence:

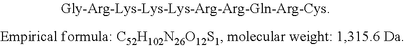

The PPT block copolymer is a high-molecular-weight biopolymer chemically synthesized in an aqueous phase, positively charged at pH 7.4-8.3. The biopolymer is based on linear PEI molecules with a molecular weight of 25 kDa. PEI combines a high ability to destabilize cell membranes with a high ability to condense DNA, protecting it from enzymatic degradation and increasing the plausibility of intact DNA entering the nucleus. PEG molecules with a molecular weight of about 1,395 Da are covalently attached to the primary amines of PEI by a chemical reaction, and the TAT peptide is attached to PEG. Modification of PEI with PEG provides a neutral surface charge, thereby reducing the aggregation of particles, reduces the toxic effect of the biopolymer, and increases the hydrophilicity and efficacy of pDNA penetration into the nuclei of transfected tumor cells. The TAT peptide, which carries a nuclear localization signal, ensures the transport of the polyplex (a complex of plasmid DNA and PPT) into the nucleus, regardless of the cell cycle stage, and facilitates the penetration of the entire construct into the cell.

In aqueous solutions, positively charged PPT block copolymer molecules form noncovalent spherical complexes (polyplexes) with negatively charged pDNA molecules due to electrostatic interactions. These complexes interact with the cell membrane and are subsequently taken up by endocytosis. The PPT-pDNA complex within the endosome is able to buffer the endosomal vesicle, which leads to its swelling and lysis, followed by the release of pDNA into the cytoplasm. Further, the PPT-pDNA complexes are transported to the nucleus along the cytoskeletal pathways. In the nucleus, the polyplex decondenses, separating pDNA from PPT. The genes contained in the pCMV-HSVtk-hGM-CSF pDNA are transcribed and translated, which leads to the synthesis of two therapeutic proteins in the cell, namely: herpes simplex virus thymidine kinase and human granulocyte-macrophage colony-stimulating factor.

In preferred embodiments of the present invention, the BC PPT is characterized by a PEG/PEI molar ratio of about 1.0 to 3.0 and a TAT/PEI molar ratio of about 0.8 to 1.5. In the most preferred embodiments of the present invention, the molar ratio of PEG/PEI is from about 2.0 to 2.5.

For efficient transfection of tumor cells, the optimal ratio of PPT and pDNA molecules in the polyplex, expressed as a ratio of N (the number of nitrogen atoms) to P (the number of phosphorus atoms), was experimentally selected equal to about 20 to 40, preferably about 30. Analysis of inorganic phosphorus in accordance with SP 14 (State Pharmacopoeia of the Russian Federation), GPM.1.7.2.0018.15 (Analysis of nucleic acids by the Spirin method in biological medicinal products) is carried out for quantitative DNA analysis.

In the medicinal product of the present invention, the PEG-PEI-TAT peptide block copolymer is contained in an amount of approximately 0.63-0.74 mg (on the PEI basis).

### Pharmaceutically Acceptable Additives

As at least one pharmaceutically acceptable additive in the manufacture of the first component containing pCMV-HSVtk-hGM-CSF plasmid DNA, a pharmaceutically acceptable buffer is used which provides a physiologically acceptable pH value of the solution in the range from 7.0 to 8.0. It is preferable to use HEPES (4-(2-hydroxyethyl)-1-piperazineethanesulfonic acid) as a buffer, which, in comparison with other buffer systems (phosphate and carbonate), maintains a given pH value much better with minimal cytotoxicity. HEPES is included in the State Pharmacopoeia of the Russian Federation XIV edition (SP 14) as a component of the buffer solution (GPM.1.3.0003.15 Buffer solutions, Buffer (HEPES) solution pH 7.5, vol. 1, page 1808). According to SP 14, buffer solutions are used, among other things: (1) to achieve isotonicity in the preparation of liquid dosage forms; and (2) to maintain the stability of dosage forms (State Pharmacopoeia of the Russian Federation XIV edition, 2018).

Also, cryoprotectants such as mono- and disaccharides (lactose, glucose or sucrose) are added to DNA to prevent loss of its transfection activity prior to the solution freezing or lyophilization process. Preferably, glucose (dextrose) is used to make the first component containing the pCMV-HSVtk-hGM-CSF plasmid DNA. Glucose can be used both in an anhydrous form and in the form of monohydrate, and the content of glucose in the medicinal product should be approximately 100 mg based on of the anhydrous form.

As at least one pharmaceutically acceptable additive in the manufacture of the second component containing BC PPT, a pharmaceutically acceptable buffer is used, which provides a physiologically acceptable pH value of the solution in the range from 7.1 to 8.2. Preferably, the borate buffer is used as such buffer, involving sodium tetraborate. Sodium tetraborate can be represented as an anhydrous form, or any of its known crystalline forms, for example, decahydrate (sodium tetraborate decahydrate). In this case, the content of sodium tetraborate in the medicinal product should be approximately 2.38 mg based on the anhydrous form.

The present invention provides a medicinal product for the treatment of solid cancers. The tissue structure of solid tumors contains interdependent tissue compartments, including parenchyma (cancer cells) and supporting stromal cells, which can provide a supportive microenvironment which cancer cells are distributed among. Solid malignancies include, but are not limited to, sarcomas, carcinomas, and lymphomas, including lung cancer, breast cancer, prostate cancer, colon cancer, rectal cancer, and bladder cancer. In some embodiments, the cancer is selected from lung cancer, pancreatic cancer, melanoma, fibrosarcoma, sarcoma, head and neck cancer (including, for example, head and neck squamous cell carcinoma—HNSCC).

Below are examples of implementation, which serve to illustrate the claimed invention, but are not intended to limit the scope of legal protection. The scope of legal protection is determined solely by the attached claims.

### Example 1. Isolation, Collection and Storage of Bacterial Biomass

Biomass, i.e. the cell sediment is separated from the culture liquid in a floor high-speed batch centrifuge with a fixed-angle rotor. To do this, portions of 800 ml of the culture suspension are added to centrifuge bottles using a graduated cylinder. The bottles are alternately weighed on the balance, the latter is calibrated by the weight of the heaviest of the six glasses and the difference in weight of the heaviest filled bottle and the rest ones is adjusted one-by-one to (0.0±0.5) g by adding the culture suspension with a Pasteur pipette. The bottles are hermetically closed with caps and placed in the rotor. Centrifugation is carried out for 15 min at a set speed of 10,000 rpm and the temperature in the centrifuge working chamber of 4° C. The culture liquid supernatant is poured into a container and transferred for decontamination by autoclaving. The bottles with the sediment are refilled with the culture suspension and the centrifugation is repeated. During the last cycle of centrifugation, purified water is used to adjust the weight of the bottles in case of a shortage of the culture suspension. At the end of the last centrifugation cycle, the bottles are placed neck down on a sheet of filter paper and incubated for (5±1) min to remove residual liquid. A biomass sample is taken with a metal spatula for authenticity control by agarose gel electrophoresis. The resulting biomass is transferred with a steel spatula into a container with a cap pre-weighed on a balance, the filled container is closed, re-weighed, a label of the established form is pasted, placed into a protective plastic bag with a Zip-lock fastener and stored in a low-temperature refrigerator at a temperature below −65° C.

### Example 2. Obtaining a Clarified Lysate of Bacterial Biomass

The procedure for obtaining a clarified lysate consists of suspending bacterial cells, cell lysis under alkaline conditions, and clarification of the lysate.

The clarified lysate is obtained in three cycles. Each cycle includes: suspending the biomass, biomass lysis, and lysate clarification.

### Suspending the Biomass

Processing of 600 g of biomass requires three lysis cycles of 200 g of biomass each. To re-suspend each 200 g of biomass, 2 liters of solution I are consumed. Solution I includes tris-(hydroxymethyl)-aminomethane, disodium ethylenediaminetetraacetate, and glucose monohydrate.

The container with the biomass is thawed (if necessary) at a temperature of +5±3° C. until the contents is completely defrosted. 200 g of the thawed biomass are weighed on a scale, 2.0 L of solution I are added, and the container is put to stir on a magnetic stirrer until a homogeneous suspension without visible cell clumps is obtained.

### Cell Lysis

For the lysis of each 200 g of biomass of the cell suspension obtained at the suspending step, 2 L of solution II are added. The resulting cell suspension is poured into a container with a screw cap and 2.0 L of solution II are poured into, stirred by turning over for 1 min, and the solution is incubated for 5 min.

Solution II is prepared as follows. 1.5 L of WFI is poured into a container, 16.00 g of sodium hydroxide and 20.00 g of sodium dodecyl sulfate are added, and the mixture is stirred with a magnetic stirrer until the reagents are completely dissolved. The volume of the solution is brought to 2.0 L with water for injection (WFI).

### Lysate Clarification

For clarification of the lysate from 200 g of biomass, 2 L of solution III are used.

2.0 L of solution III is poured into the container with the lysate obtained at the lysis step and mixed by vigorously inverting several times. Solution III is prepared as follows. 0.9 L of WFI is poured into a container, 588.9 g of potassium acetate is added, and the mixture is stirred with a magnetic stirrer until the reagents are completely dissolved. The pH is adjusted to 5.5 by adding 0.3 L of acetic acid solution. The volume of the solution is brought to 2.0 L with WFI. The buffer solution is filtered into a container using a filter. The solution is then incubated for 10 min. Cellular debris is removed by centrifugation at 15,000 rpm for 30 min or by filtration through a thick filter paper. The clarified cell lysate is concentrated by ultrafiltration using a glass fiber filter.

### Example 3. Isolation of Plasmid DNA from Biomass

As a primary chromatographic purification of supercoiled plasmid DNA, a gel filtration chromatography step is used, which allows ridding the plasmid solution of ribonucleic acid impurities. Purification is carried out on a column filled with Sepharose 6FF sorbent using an Akta Pilot chromatographic system. Approximately four gel filtration cycles are required to obtain the required amount of the final product and to purify the entire volume of the concentrated clarified lysate: two cycles per each ultrafiltration cycle. After each cycle, the sorbent is re-equilibrated, and every two cycles it is regenerated followed by re-equilibration.

The affinity chromatography step is used to purify the supercoiled ring form of the target DNA from the relaxed one. Purification is carried out on a column filled with Plasmid Select Xtra sorbent using an Akta Pilot chromatographic system.

Purification of the target combined fraction obtained from one step of gel filtration requires 2-3 purification cycles on an affinity sorbent. To purify the entire volume of the pDNA fraction from the gel filtration step and to obtain the required amount of the target substance, a total of approximately six cycles of affinity chromatography is required. The sorbent is regenerated and re-equilibrated after each cycle.

For the final purification of the supercoiled circular form of DNA from impurities, an anion exchange chromatography step is used. Purification is carried out on a column filled with Source 30Q sorbent using an Akta Pilot chromatographic system. Regeneration is carried out after each cycle; the sorbent is depyrogenated and re-equilibrated before each cycle. Approximately two cycles of chromatographic purification are required to process the entire volume of the target product.

### Example 4. Filling the Solution of Plasmid Deoxyribonucleic Acid with Excipients (First Component) into Bottles and Capping

In zone A of a grade B clean room with the laminar air flow turned on, the product is filled in accordance with the calculated filling dose (100-102.5%) into 2R bottles (in cassettes) made of light glass of the first hydrolytic class, which enter the filling and capping room through a pass-through dry oven. The first (3±1) bottles are used to set the filling dose (volume). Filling is carried out by means of a peristaltic dosing pump and a sterile silicone filling hose with an inner diameter of 3.2 mm. Dose control is carried out by weight at the beginning, middle, and end of bottling. The contents of the first three bottles (corresponding to approximately 0.0036 L), filled incompletely or with a dose error, are transferred for disposal or recycling. Filling is checked by weight method, at least five times during operation. The bottling of 0.594 L of solution is carried out. 0.015 L of the solution remains in the line (hoses), which is poured into a sterile collection tank and then transferred for disposal or recycling.

### Example 5. Filling the Solution of PPT Block Copolymer with Excipients (Second Component) into Bottles and Capping

In zone A of a grade B clean room with the laminar air flow turned on, the product is filled in accordance with the calculated filling dose (100-102.5%) into 2R bottles (in cassettes) made of light glass of the first hydrolytic class, which enter the filling and capping room through a pass-through dry oven. The first (3±1) bottles are used to set the bottling dose (volume) of 1.1-1.12. Filling is carried out by means of a peristaltic dosing pump and a sterile silicone filling hose with an inner diameter of 3.2 mm. Dose control is carried out by weight at the beginning, middle, and end of bottling.

The contents of the first three bottles (corresponding to approx. 0.0033 L), filled incompletely or with dose errors, are transferred for disposal or recycling. Filling is checked by weight method, at least five times during operation.

Throughout the bottling, each bottle is instantly covered with a stopper.

During operation, the quality of bottle capping is monitored (visually).

0.0015 L of the solution remains in the line (hoses), which is poured into a sterile collection tank and then transferred for disposal or recycling.

Filled and capped bottles are placed into a cassette and transferred to the step of flip cap sealing.

### Example 6. Medicinal Product for Combination Gene-Directed Enzyme Prodrug Therapy—Immune Therapy of Solid Malignant Neoplasms, 2 ml (Variant 1)

In the manufacture of the medicinal product, raw materials are used which have passed incoming control for compliance with regulatory and technical documentation:
plasmid deoxyribonucleic acid substance
PPT block copolymer substance
Sodium tetraborate decahydrate
HEPES 1 M solution
Dextrose monohydrate 40% solution (on anhydrous glucose basis).
   First Component—Solution of pCMV-HSVtk-hGM-CSF pDNA for Preparing Solution for Injection, 1 ml.

| | | | |
|---|---|---|---|
| Active ingredient: | Plasmid | 160.0 | μg |
| | deoxyribonucleic acid | | |
| Excipients: | Dextrose | 100 | mg |
| | HEPES | 2.38 | mg |

| | |
|---|---|
| Water for injection | to a final volume of 1.0 ml |

DESCRIPTION. Frozen solution. Dense hardened mass of whitish color. After thawing: colorless transparent solution without visible inclusions.

STORAGE. At temperatures not higher than −18° C. No storage of the thawed product is allowed. Re-freezing is not allowed.

### Second Component—Solution of Polyethylene Glycol-Polyethyleneimine-TAT Peptide Block Copolymer, for Preparing Solution for Injection, 1 ml

| | | |
|---|---|---|
| Active ingredient: | PPT block copolymer | 0.69 mg |
| Excipients: | Sodium tetraborate | 2.38 mg |
| | Water for injection | to a final volume of 1.0 ml |

DESCRIPTION. Frozen solution. Dense hardened mass of whitish color. After thawing: colorless transparent solution without visible inclusions.

STORAGE. At temperatures not higher than −18° C. No storage of the thawed product is allowed. Re-freezing is not allowed.

### Preparation of the Ready-to-Use Dosage Form of the Medicinal Product (Variant 1)

To prepare the ready-to-use dosage form “solution for injection” of the medicinal product, it is necessary to thaw the contents of the bottles containing the first and second components. Then it is necessary to carefully mix the contents of the bottles, making circular movements and gently rocking them. When preparing the ready-to-use dosage form of the medicinal product, vigorous mixing or shaking should be avoided. After thawing and mixing, the contents of the bottle containing the first component are transferred to the bottle with the second component and mixed by rocking. The mixture is kept at room temperature for 30 min. The ready-to-use medicinal product should be used within 2 hours from the time of preparation. Care should be taken to ensure the sterility of the prepared solution, as the medicinal product contains neither preservatives nor bacteriostatic substances.

### Example 7. Obtaining a Lyophilized Form of the First Component Containing Plasmid DNA

The bottles containing the first component of the medicinal product as described in Example 6 are subjected to lyophilization in a freeze-dryer according to the program shown in Table 1.

TABLE 1
Conditions for lyophilization of the first
component of the medicinal product

| | | Shelf | Chamber |
|---|---|---|---|
| Phase | Duration, h | temperature, ° C. | pressure, mbar |
| Freezing | 2 | +20-−40 | 1,000 |
| Delay | 2 | −40 | 1,000 |
| Primary drying | 2 | +25 | 1,000-0.4 |
| | 12 | +25 | 0.4 |
| Secondary drying | 8 | +25 | 0.2 |

After completion of the drying program, the bottles are hermetically sealed in an argon atmosphere in a freeze-dryer. Bottles are stored in a place protected from light at temperatures not exceeding 4° C.

### Example 8. Medicinal Product for Combination Gene-Directed Enzyme Prodrug Therapy-Immune Therapy of Solid Malignant Neoplasms, 2 ml (Variant 2)

Composition of the First Component, Lyophilized pCMV-HSVtk-hGM-CSF Plasmid DNA for Preparing Solution for Injection

| Active substance: | | |
|---|---|---|
| pCMV-HSVtk-hGM-CSF supercoiled plasmid DNA | 0.16 | mg |
| Excipients: | | |
| Dextrose | 100 | mg |
| HEPES | 2.38 | mg |

OBTAINING THE FIRST COMPONENT. The pCMV-HSVtk-hGM-CSF plasmid DNA solution prepared as described in Example 3 is lyophilized as described in Example 7.

DESCRIPTION Lyophilisate. A mass of whitish color. After reconstitution by adding 1.0 ml of water for injection: colorless transparent solution without visible inclusions.

STORAGE. At temperatures not higher than 4° C.

### Composition of the Second Component, PPT Solution, 1 ml for Preparing Solution for Injection.

| | |
|---|---|
| Active substance: | |
| PPT | 0.69 mg |
| Excipients: | |
| Sodium tetraborate | 2.38 mg |
| Water for injections | to a final volume of 1.0 ml |

### Preparation of the Ready-to-Use Dosage Form of the Medicinal Product (Variant 2)

To prepare the ready-to-use dosage form “solution for injection” of the medicinal product, it is necessary to thaw the contents of the bottle containing the second component. 1.1 ml of water for injection should be added to the bottle with the first component, and gently mixed until the lyophilisate is completely dissolved. Then it is necessary to carefully mix the contents of the bottles, making circular movements and gently rocking them. When preparing the ready-to-use dosage form of the medicinal product, vigorous mixing or shaking should be avoided. After thawing and mixing, the contents of the bottle containing the first component is transferred to the bottle with the second component and mixed by rocking. The mixture is kept at room temperature for 30 min. The ready-to-use medicinal product should be used within 2 hours from the time of preparation. Care should be taken to ensure the sterility of the prepared solution, as the medicinal product contains neither preservatives nor bacteriostatic substances.

Composition of the Ready-to-Use Dosage Form “Solution for Injection” of the Medicinal Product after Mixing the First and Second Components (Variant 2)

| Active substances: | | |
|---|---|---|
| pCMV-HSVtk-hGM-CSF | 0.16 | mg |
| supercoiled plasmid DNA | | |
| PPT | 0.69 | mg |
| Excipients: | | |
| Dextrose | 100 | mg |
| HEPES | 2.38 | mg |
| Sodium tetraborate | 2.38 | mg |

| | |
|---|---|
| Water for injections | to a final volume of 2.0 ml |

Description. Transparent colorless liquid without foreign inclusions.

### Example 9. Cytotoxic Test to Assess the Specific Activity of the Medicinal Product

Specific activity is checked by the formation of a complex of the pCMV-HSVtk-hGM-CSF plasmid DNA (first component) with the PPT block copolymer (second component) in a cytotoxic test on C-26 cell line (CT26.WT murine colorectal adenocarcinoma, ATCC® CRL-2638™) in vitro. The method for evaluating the activity of the medicinal product is based on the ability of the medicinal product to cause the death of C-26 cancer cells transfected with the medicinal product in the presence of ganciclovir. The number of viable cells should not be more than 50% or 20% at a ganciclovir concentration of 2 μM or 12.5 μM in the growth medium, respectively.

All work is performed under sterile conditions under laminar air flow and using sterile test tubes and pipettes.

Cells are grown in RPMI 1640 nutrient medium (Gibco, USA). Cells are grown in a CO₂ incubator at 37° C. in an atmosphere containing 5% CO₂. When the monolayer confluence reaches 90% (assessed visually), the cells are sub-cultured in the RPMI1640/antibiotic nutrient medium at a ratio of 1:10. For the test, cells from passages 3-10 are used.

20-24 hours before the test, C-26 cells are sub-cultured in a 12-well plate.

### Carrying Out Transfection of C-26 Cells

The wells of a 12-well plate (e.g., Corning-353043, Corning, USA) are inoculated with (2−3)·10⁵ cells in 2 ml of RPMI 1640 medium (without antibiotic) and incubated in a CO₂ incubator for 24 hours. Before adding the solutions of complexes for transfection and the test sample of the medicinal product, the medium is removed from the wells of the plate with a pipette, without touching the bottom and walls of the well. After adding the solutions of the complexes and the test sample of the medicinal product, the plate is incubated in a CO₂ incubator for 48 hours.

### Counting the Number of Viable Cells

The plate is removed from the CO₂ incubator, the medium is removed from all wells with a pipette, and the wells are washed with 2 ml of phosphate buffer PBS. 0.2 ml of trypsin-EDTA solution is added to each well and incubated in the CO₂ incubator for 2 min. 1 ml of RPMI 1640/antibiotic medium is added, the cells are resuspended with a pipette and transferred to sterile microtubes.

The concentration of viable cells is assayed using trypan blue.

### Counting the Number of Fluorescent Cells

To assess the efficacy of transfection of C-26 cells transfected with Lipofectamine 3000 and PPT containing the pEGFP-N1 indicator plasmid, transfected cells are counted using a flow cytometer. A sample of Ccl and CGFP (cells transfected with pEGFP-N1—Lipofectamine complex and pEGFP—N1-PPT complex, respectively) in an amount of 50 μl is added to microtubes containing 50 μl of an assay buffer containing 10 mM phosphate buffer, pH 7.4 with 2.5% BSA and 0.1% sodium azide.

Transfection efficacy is calculated as the percentage of cells with fluorescence which exceeds the autofluorescence level of non-transfected cells.

After transfection, 100 μl of transfected and non-transfected cell suspensions are added to two 96-well plates. The plates with cell suspensions are incubated in a CO₂ incubator for 24 hours. Next, a solution of ganciclovir is prepared at concentrations of 0, 0.4, 4.0, 25.0 and 100.0 μM. After incubation of cell suspensions, 100 μl of ganciclovir at various concentrations are added to the wells.

The plates with cell suspensions are incubated in a CO₂ incubator for 72 hours.

After the incubation, the medium is removed from the wells and 200 μl of ganciclovir are added at various concentrations.

The plates are incubated in a CO₂ incubator until a cell monolayer (assessed visually) is reached in the wells with 0 μM ganciclovir.

After the incubation, 100 μl of the medium is taken from each well. 20 μl of MTS reagent (Cell Titer 96® AQueous One Solution Cell Proliferation Assay (MTS), cat. No: G3582, Promega) are added to all wells. The contents of the wells are mixed on a shaker and incubated in a CO₂ incubator for 30 min.

The optical density AN in the wells is measured at a wavelength of 492 nm using a plate photometer. The value of the optical density A_{abs}N is calculated for each well, taking into account the background value according to the formula

$${A}_{abs} N = AN - {A}_{bg}$$

An average value A_{abs}Nₐᵥ for triplicates is calculated for each sample of transfected C-26 cell line cells.

The percentage of viable cells in dilutions of ganciclovir is calculated for all experimental and control samples of transfected cells using the formula:

$$\% viable cells = {A}_{abs} {N}_{av} \times CN \times 100 / {A}_{abs} {0}_{av} ,$$

where
A_{abs}Nₐᵥ is the average value of optical density from triplicates for each sample of transfected cells at various dilutions of ganciclovir,
CN is the ganciclovir concentration in the wells, and
A_{abs}0ₐᵥ is the average value of optical density from triplicates for each sample of transfected cells at a zero concentration of ganciclovir.

A test is considered significant if:
the percentage of viable cells in Ccl and CGFP negative controls and non-transfected cells exceeds 90%,

A batch of the medicinal product is considered suitable for use if the percentage of viable cells in three samples of the medicinal product from the batch does not exceed 20% in the wells with a ganciclovir concentration of 12.5 μM.

### Example 10. Determination of Characteristics of PPT (Second Component)

The PEI content in the synthesized PPT is measured spectrophotometrically, for example, using a plate reader Synergy 4 (BioTek Instruments, USA, or a similar one) at a wavelength of 620 nm. To construct a calibration curve and measure conjugate concentrations, 50 μl of the appropriate PEI or water sample, 100 μl of 40 mM Cu(CH₃COO)₂·H₂O, and 50 μl of water are added to each well of a 96-well plate. The calibration solutions are measured in triplicate. The optical density of 20 mM Cu(CH₃COO)₂·H₂O is subtracted from the obtained average optical density value. The absorption of the resulting copper complex at 620 nm depends linearly on the PEI concentration (C_{PEI}=a₁·A₆₂₀, where C_{PEI} is the concentration of PEI in the calibration sample, in μM, A₆₂₀ is the average optical density of the calibration sample minus the optical density of the sample without PEI, a₁ is the proportionality factor). The PPT concentration is taken equal to the PEI concentration in the PPT solution.

The PEG content in the synthesized PPT is measured spectrophotometrically, for example, using a Synergy 4 plate reader (BioTek Instruments, USA), or a similar one, at a wavelength of 540 nm. To do this, freshly prepared PEG calibration solutions are used with a molecular mass of 1,500 Da: 200 μM, 160 μM, 120 μM, 80 μM, 40 μM and 20 μM PEG, as well as 5% barium chloride solution and a solution of 12.3 g/l I₂ in 2% KI. 10 μl of a PEG solution with a known concentration or a sample to be measured (previously diluted to a concentration of 20 μM PEI), 10 μl of a 20 μM solution of PEI or water for an unknown sample to be measured, 180 μl of water, 50 μl of the solution of barium chloride and 25 μl of the iodine solution in potassium iodide are added to the wells of a 96-well plate, and after 10-13 minutes the absorption of the solutions is measured at 540 nm. The time is counted from the moment when the iodine solution is added. Measurements are made in triplicate. The absorbance at 540 nm is linearly dependent on the PEI concentration (C_{PEG}/C_{PEI}=a₂·A₅₄₀, where C_{PEG}/C_{PEI} is the PEG/PEI molar ratio of the calibration sample, in μM, A₅₄₀ is the average absorbance of the calibration sample minus the absorbance of the sample without PEG, a₂ is the proportionality factor).

The content of the TAT peptide in the synthesized PPT is measured fluorometrically by the content of the adduct formed by the amino groups of the TAT peptide in the presence of fluorescamine. To do this, a solution of fluorescamine in acetone (1 mg/ml) and 0.25 M phosphate buffer, pH 9.3, are prepared. The 6 mg/ml TAT peptide solution is diluted to a concentration of 100 μM. 3 ml of a mixture of the TAT peptide solution with the fluorescamine solution are prepared, such that the final concentration of TAT peptide is 0.5 μM: 15 μl of 100 μM TAT peptide solution, 60 μl of 1 mg/ml fluorescamine solution, and 2,925 μl 0.25 M phosphate buffer, pH 9.3. 3 ml of a mixture of the PEI-PEG-TAT block copolymer solution with the fluorescamine solution are prepared, such that the final concentration of PEI in the block copolymer is 1 μM. Fluorescence measurements are carried out in a glass cuvette at a fluorescence excitation wavelength of 395 nm and a fluorescence wavelength of 490 nm. The fluorescence values at each point are measured eight times and the readings are averaged. Synthesis efficacy is considered optimal when the TAT/PEI molar ratio is approximately 0.8 to 1.5.

In the course of our preliminary experiments, the PEG/PEI ratios were found, at which polyplexes provide a high efficacy of pDNA delivery to cancer cells. For all cell lines tested, the highest transfection efficacy was achieved at PEG/PEI molar ratios of about 1.0 to 3.0, more preferably at ratios of about 2.0 to 2.5, most preferably at a ratio of about 2.0.

### Example 11. Stability of the Dosage Form of the Medicinal Product (Variant 1—the First and Second Components in the Form of Frozen Solutions)

The goal of our study was to examine the stability of the physical, chemical and biological characteristics of the medicinal product of the present invention under storage conditions at a temperature of −20° C.±2° C. with an estimated shelf life of 18 months.

The first and second components were stored at a temperature of −20° C.±2° C. C packed in colorless bottles of hydrolytic class 1, hermetically sealed with rubber stoppers and aluminum flip caps.

The stability of the components and the ready-to-use dosage form of the medicinal product was assessed by a number of parameters, including: description, authenticity (restrictase analysis), authenticity (PCR with specific primers), transparency, color, mechanical inclusions, residual proteins of the producer strain, bacterial endotoxins, abnormal toxicity, sterility, and specific activity. The results obtained in the framework of this study are shown in Tables 2-4.

TABLE 2
Results of the stability study of the first component, Batch No. 040820

| | | 0 (start of | | | | | |
|---|---|---|---|---|---|---|---|
| No. | Quality indicator | storage) | 3 months | 6 months | 9 months | 12 months | 18 months |
| 1 | Description | After thawing: | After thawing: | After thawing: | After thawing: | After thawing: | After thawing: |
| | | colorless | colorless | colorless | colorless | colorless | colorless |
| | | transparent | transparent | transparent | transparent | transparent | transparent |
| | | liquid without | liquid without | liquid without | liquid without | liquid without | liquid without |
| | | visible | visible | visible | visible | visible | visible |
| | | inclusions | inclusions | inclusions | inclusions | inclusions | inclusions |
| 2 | Authenticity, | Complies | Complies | Complies | Complies | Complies | Complies |
| | Restrictase analysis | | | | | | |
| 3 | Authenticity, PCR | Complies | Complies | Complies | Complies | Complies | Complies |
| | with specific primers | | | | | | |
| 4 | Transparency | Complies | x | Complies | x | Complies | x |
| 5 | Color | Complies | x | Complies | x | Complies | x |
| 7 | Mechanical | Complies | Complies | Complies | x | Complies | x |
| | inclusions, visible | | | | | | |
| 10 | Residual proteins of | Complies | x | x | Complies | Complies | x |
| | the producer strain | | | | | | |
| 11 | Bacterial endotoxins | Complies | x | Complies | Complies | Complies | Complies |
| 12 | Abnormal toxicity | Non-toxic | x | Non-toxic | x | Non-toxic | x |
| 13 | Sterility | Sterile | Sterile | Sterile | x | Sterile | x |

TABLE 3
Results of the stability study of the second component, Batch No. 040820

| | | 0 (start of | | | | | |
|---|---|---|---|---|---|---|---|
| No. | Quality indicator | storage) | 3 months | 6 months | 9 months | 12 months | 18 months |
| 1 | Description | After thawing: | After thawing: | After thawing: | After thawing: | After thawing: | After thawing: |
| | | colorless | colorless | colorless | colorless | colorless | colorless |
| | | transparent | transparent | transparent | transparent | transparent | transparent |
| | | liquid without | liquid without | liquid without | liquid without | liquid without | liquid without |
| | | visible | visible | visible | visible | visible | visible |
| | | inclusions | inclusions | inclusions | inclusions | inclusions | inclusions |
| 2 | Authenticity | Complies | Complies | Complies | Complies | Complies | Complies |
| 3 | Transparency | Complies | x | Complies | x | Complies | x |
| 4 | Color | Complies | x | Complies | x | Complies | x |
| 5 | Mechanical | Complies | Complies | Complies | x | Complies | x |
| | inclusions | | | | | | |
| 6 | Specific activity | Complies | Complies | Complies | Complies | Complies | Complies |
| 7 | Bacterial endotoxins | <25 EU/mg | Complies | Complies | x | Complies | x |
| 8 | Sterility | Sterile | Sterile | Sterile | x | Sterile | x |

TABLE 4
Results of the stability study of the ready-to-use dosage form “solution for injection”
of the medicinal product after opening and mixing the first and second components, Batch No. 040820

| | | 0 (start of | | | | | |
|---|---|---|---|---|---|---|---|
| No. | Quality indicator | storage) | 3 months | 6 months | 9 months | 12 months | 18 months |
| 1 | Description | After mixing: | After mixing: | After mixing: | After mixing: | After mixing: | After mixing: |
| | | colorless | colorless | colorless | colorless | colorless | colorless |
| | | transparent | transparent | transparent | transparent | transparent | transparent |
| | | solution without | solution without | solution without | solution without | solution without | solution without |
| | | visible | visible | visible | visible | visible | visible |
| | | inclusions | inclusions | inclusions | inclusions | inclusions | inclusions |
| 2 | Authenticity | Complies | Complies | Complies | Complies | Complies | Complies |
| 3 | Specific activity, | Complies | Complies | Complies | Complies | Complies | Complies |
| | cytotoxic test | | | | | | |

Thus, in our study of stability, it was found that storage conditions at a temperature of −20° C.±2° C. ensure the stability of the quality indicators of the first and second components, as well as of the ready-to-use solution for injection obtained after mixing the first and second components, for a shelf life of 18 months. The established shelf life of the medicinal product is at least 18 months.

### Example 12. Stability of the Dosage Form of the Medicinal Product (Variant 2—the First Component in the Form of a Lyophilisate, the Second Component in the Form of a Frozen Solution)

The goal of the study was to examine the stability of the physical, chemical and biological characteristics of the medicinal product under storage conditions of the first component at a temperature of 4° C. and the second component at a temperature of −20° C.±2° C. during the expected shelf life of 24 months.

The first component was stored at 4° C. packed in hydrolytic class 1 colorless bottles sealed with rubber stoppers and aluminum flip caps. The second component was stored at a temperature of −20° C.±2° C. packed in colorless bottles of hydrolytic class 1, hermetically sealed with rubber stoppers and aluminum flip caps.

The ready-to-use dosage form was prepared as described above in Example 8.

Stability was studied by measuring a number of parameters, including the specific activity of the ready-to-use dosage form of the drug (variant 2) after 1 day, 2 weeks, 3 months, 6 months, 12 months and 18 months after the start of the experiment. Specific activity was measured as described above in Example 9 (“Cytotoxic test to assess the specific activity of the medicinal product”). As a result of the experiments, it was shown that the medicinal product (variant 2) retains its biological activity for at least 18 months.

### Example 13. Study of the Antitumor Activity of the Medicinal Product

The antitumor effect of the medicinal product was studied in xenograft models of human tumors in nude immunodeficient mice obtained by inoculation of human tumor cells: KB oral mucosal carcinoma, HEp2 human laryngopharyngeal carcinoma, HT29 colorectal adenocarcinoma, and HeLa cervical adenocarcinoma. Animals (females, n=9-12 animals per group) were treated using an effective regimen. Treatment was started on day 11 (KB, HT29, HEp2) and on day 13 (HeLa) of tumor growth, when the tumor volume was approximately 100±10 mm³.

The summarized results of the study are presented in Table 5.

TABLE 5
Antitumor activity of the combination “medicinal
product of the present invention + ganciclovir”
in subcutaneous human tumor xenograft models

| | TGI, | T/C, | T − C, | Median |
|---|---|---|---|---|
| Human tumor xenograft | % | % | days | survival, days |
| KB oral mucosal carcinoma | 82* | 18 | 32.0 | 82.0** |
| HT29 colon adenocarcinoma | 89* | 11* | 49.0 | 89.1** |
| HeLa cervical adenocarcinoma | 57 | 43 | 21.8 | 71.1 |
| HEp human laryngopharyngeal | 46 | 64 | 16.6 | 74.8** |
| carcinoma 2 | | | | |

Note:
TGI (tumor growth inhibition, %) and T/C (tumor inhibition degree, %) are given on the 28^{th} day after the end of treatment (for KB), on the 30^{th} day (for HeLa and HEp2), or on the 38^{th} day (for HT29).
*high antitumor activity (efficacy criteria: TGI ≥ 70%, T/C ≤ 15%, ILE ≥ 50% (increased life expectancy, %).
**statistically significant difference from the group of animals which were injected with PBS (control group) (p < 0.05; Student's t-test).
T − C, tumor growth retardation.

As follows from the results presented in Table 5, a high (according to the efficacy criteria) antitumor effect was detected in nude mice with KB human oral mucosa carcinoma: their TGI increased with the increase in the observation period and amounted to 66-82% on days 46-60 after tumor cells inoculation, the tumor growth delay was 32 days, the median survival of animals in the experimental and control groups differed by 24 days (82 and 58 days, respectively).

A significant antitumor effect was also observed in relation to HT29 human colon adenocarcinoma: TGI—more than 70% during 32-67 days of observation, with the maximum TGI (89%) observed at the latest day 67; a delay in tumor growth was also observed (49 days). The median survival of animals in the experimental group exceeds that of animals in the control group by 14 days (89 and 75 days, respectively).

The efficacy of the “medicinal product of the present invention+ganciclovir” combination in HeLa xenograft mice was significantly lower than for HT29 and KB tumor models. The lowest antitumor treatment efficacy was noted in the HEp2 xenograft model in immunodeficient animals. At the same time, however, the median survival of animals in the experimental and control groups differed significantly by 13.6 days (74.8 and 61.2 days, respectively).

The results of the in vivo studies using animal models of a number of human solid malignancies have revealed that KB oral mucosal carcinoma and HT29 human colorectal carcinoma are the most susceptible.

1. A medicinal product for the treatment of solid malignant neoplasms by the method of combination gene-directed enzyme prodrug therapy—immune therapy, containing two hermetically sealed containers, wherein:
the first container contains the first component containing pCMV-HSVtk-hGM-CSF supercoiled plasmid DNA in a therapeutically effective amount, at least one pharmaceutically acceptable additive and water,
the second container contains the second component containing the PEG-PEI-TAT peptide block copolymer in a therapeutically effective amount, at least one pharmaceutically acceptable additive and water,
wherein the contents of each of the containers is sterile, and the containers are stored at a temperature not higher than −18° C.

2. The medicinal product according to claim 1, wherein:
the first component contains:
pCMV-HSVtk-hGM-CSF supercoiled plasmid DNA—approximately 150-170 μg,
dextrose—approximately 100 mg,
HEPES—approximately 2.38 mg, and
water—to a final volume of 1.0 ml,
the second component contains:
PEG-PEI-TAT peptide block copolymer—approximately 0.63-0.74 mg (on the PEI basis),
sodium tetraborate—approximately 2.38 mg, and
water—to a final volume of 1.0 ml.

3. The medicinal product according to claim 1, wherein the ready-to-use solution for injection is obtained by thawing the contents of the said first and second containers and mixing them.

4. The medicinal product according to claim 1, wherein the ready-to-use solution for injection is administered intratumorally.

5. The medicinal product of claim 1, wherein the solid cancers are selected from the group comprising: lung cancers, pancreatic cancers, melanoma, fibrosarcoma, sarcoma, head and neck cancers.

6. The medicinal product of claim 1, wherein the PEG-PEI-TAT peptide block copolymer has a PEG/PEI molar ratio of about 1.0 to 3.0 and a TAT/PEI molar ratio of about 0.8 to 1.5.

7. The medicinal product of claim 6, wherein the PEG-PEI-TAT peptide block copolymer has a PEG/PEI molar ratio of about 2.0 to 2.5.

8. The medicinal product according to claim 1, wherein:
the first component contains:
pCMV-HSVtk-hGM-CSF supercoiled plasmid DNA—approximately 160 g,
dextrose—approximately 100 mg,
HEPES—approximately 2.38 mg, and
water—to a final volume of 1.0 ml,
the second component contains:
PEG-PEI-TAT peptide block copolymer—approximately 0.69 mg (on the PEI basis),
sodium tetraborate—approximately 2.38 mg, and
water—to a final volume of 1.0 ml,
wherein the PEG-PEI-TAT peptide block copolymer has a PEG/PEI molar ratio of approximately 2.0.

9. A medicinal product for the treatment of solid malignant neoplasms by the method of combination gene-directed enzyme prodrug therapy—immune therapy, containing two hermetically sealed containers, wherein:
the first container contains the first component, which is a lyophilizate containing pCMV-HSVtk-hGM-CSF supercoiled plasmid DNA in a therapeutically effective amount and at least one pharmaceutically acceptable additive,
the second container contains the second component containing the PEG-PEI-TAT peptide block copolymer in a therapeutically effective amount, at least one pharmaceutically acceptable additive and water,
wherein the contents of each of the containers is sterile, and the first container is stored at a temperature not higher than 4° C., and the second container is stored at a temperature not higher than −18° C.

10. The medicinal product according to claim 9, wherein:
the first component, which is a lyophilisate, has been obtained by lyophilization of the solution containing:
pCMV-HSVtk-hGM-CSF supercoiled plasmid DNA—approximately 150-170 μg,
dextrose—approximately 100 mg,
HEPES—approximately 2.38 mg, and
water—to a final volume of 1.0 ml,
the second component contains:
PEG-PEI-TAT peptide block copolymer—approximately 0.63-0.74 mg (on the PEI basis);
sodium tetraborate—approximately 2.38 mg, and
water—to a final volume of 1.0 ml.

11. The medicinal product according to claim 9, wherein the ready-to-use solution for injection is obtained by reconstituting the contents of the first container by adding 1.0 ml of water, thawing the contents of the second container and mixing them.

12. The medicinal product according to claim 9, where the ready-to-use solution for injection is administered intratumorally.

13. The medicinal product of claim 9, wherein the solid cancers are selected from the group comprising: lung cancers, pancreatic cancers, melanoma, fibrosarcoma, sarcoma, head and neck cancers.

14. The medicinal product of claim 9, wherein the PEG-PEI-TAT peptide block copolymer is characterized by a PEG/PEI molar ratio of about 1.0 to 3.0 and a TAT/PEI molar ratio of about 0.8 to 1.5.

15. The medicinal product of claim 14, wherein the PEG-PEI-TAT peptide block copolymer has a PEG/PEI molar ratio of about 2.0 to 2.5.

16. The medicinal product according to claim 9, wherein
the first component, which is a lyophilisate, has been obtained by lyophilization of the solution containing:
pCMV-HSVtk-hGM-CSF supercoiled plasmid DNA—approximately 160 g,
dextrose—approximately 100 mg,
HEPES—approximately 2.38 mg, and
water—to a final volume of 1.0 ml,
the second component contains:
PEG-PEI-TAT peptide block copolymer—approximately 0.69 mg (on the PEI basis);
sodium tetraborate—approximately 2.38 mg, and
water—to a final volume of 1.0 ml,
wherein the PEG-PEI-TAT peptide block copolymer has a PEG/PEI molar ratio of approximately 2.0.
